(19) United States
(12) Patent Application Publication
(10) Pub. No.: US 2023/0320603 A1
(43) Pub. Date: Oct. 12, 2023

(54) BIOLOGICAL DETECTION DEVICE, BIOLOGICAL DETECTION METHOD, AND PROGRAM

(71) Applicant: Data Solutions, Inc., Tokyo (JP)

(72) Inventors: Tomoaki OTSUKI, Yokohama-shi, Kanagawa (JP); Kohei YAMAMOTO, Yokohama-shi, Kanagawa (JP)

(73) Assignee: Data Solutions, Inc., Tokyo (JP)

(21) Appl. No.: 18/042,442

(22) PCT Filed: Aug. 18, 2021
(86) PCT No.: PCT/JP2021/030151

§ 371 Date: Feb. 21, 2023

(30) Foreign Application Priority Data
Aug. 21, 2020 (JP) 2020-140401

(51) Int. Cl. A61B 5/024; A61B 5/05; A61B 5/00; G01S 7/41

(52) U.S. Cl. CPC A61B 5/024; G01S 7/415; A61B 5/7278; A61B 5/05

(57) ABSTRACT

A biological detection device includes a signal acquirer that acquires a signal including a heartbeat, an analyzer that performs a frequency analysis of the signal to generate a spectrogram, an integrated value calculator that integrates energy of a heartbeat frequency component, which is a frequency component corresponding to the heartbeat, among frequency components of the signal shown by the spectrogram and calculates an integrated value, a peak specifier that specifies peaks, which are maximum values, in the integrated value, a peak set generator that combines, among the peaks, a first peak and a second peak, which occurs in a later time than the first peak, and generates a peak set, and a selector that selects, from the peak set, a sequence having highest likelihood in a maximum likelihood estimation method.

### TECHNICAL FIELD

The present invention relates to a biological detection device, a biological detection method, and a program.

### BACKGROUND ART

There has been known a technique for measuring biological information such as a heart rate with wearable equipment and notifying a user if the biological information has an abnormality (for example, Non-Patent Literature 1).

In a watching system, first, observation equipment such as a nurse call button, a human sensor, a Doppler sensor, a heartbeat meter, a respiration measuring instrument, a thermo-camera, a manometer, a clinical thermometer, an illuminance meter, a thermometer, or a hygrometer is connected to an observed person such as an aged person. The watching system acquires observation information of the observed person in this way. The watching system determines, based on the observation information, whether an emergency alarm condition is satisfied and, if an emergency occurs, performs an emergency alarm. There has been known such a watching system that uses a vital sensor (for example, Patent Literature 1).

### CITATION LIST

### Non-Patent Literature

Non-Patent Literature 1: “Heart Rate. Its meaning and a display method in an Apple Watch (registered trademark)”, [online], Jan. 21, 2020, [searched on Mar. 2, 2020], Internet <URL: http://support.apple.com/ja-jp/HT204666>

### Patent Literature

Patent Literature 1: Japanese Patent Application Laid-Open No. 2017-151755

### SUMMARY OF INVENTION

### Technical Problem

In view of the fact that it is difficult to accurately measure a heartbeat in the related art, an object of the present invention is to accurately measure a heartbeat.

### Solution to Problem

A requirement of a biological detection device is to include:

a signal acquirer that acquires a signal including a heartbeat;

an analyzer that performs a frequency analysis of the signal to generate a spectrogram;

an integrated value calculator that integrates energy of a heartbeat frequency component, which is a frequency component corresponding to the heartbeat, among frequency components of the signal shown by the spectrogram and calculates an integrated value;

a peak specifier that specifies peaks, which are maximum values, in the integrated value;

a peak set generator that combines, among the peaks, a first peak and a second peak, which occurs in a later time than the first peak, and generates a peak set; and

a selector that selects, from the peak set, a sequence having highest likelihood in a maximum likelihood estimation method.

### Advantageous Effects of Invention

According to the disclosed technique, it is possible to accurately measure a heartbeat.

### BRIEF DESCRIPTION OF DRAWINGS

FIG. 1 is a diagram showing an overall configuration example in a first embodiment.

FIG. 2 is a diagram showing an example of a Doppler radar.

FIG. 3 is a diagram showing an example of a biological detection device.

FIG. 4 is a diagram showing an overall processing example in the first embodiment.

FIG. 5 is a diagram showing an example of a first signal.

FIG. 6 is a diagram showing an example of a spectrogram.

FIG. 7 is a diagram showing a calculation result example of an integrated value.

FIG. 8 is a diagram showing a specific example of peaks.

FIG. 9 is a diagram showing an example of selection by a maximum likelihood estimation method.

FIG. 10 is a diagram showing an example of a heartbeat interval.

FIG. 11 is a diagram showing an example of a histogram of the difference between adjacent RRIs.

FIG. 12 is a diagram showing a functional configuration example in the first embodiment.

FIG. 13 is a diagram showing a generation example of segment data.

FIG. 14 is a diagram showing an output example of a probability value by a learned model.

FIG. 15 is a diagram showing an experiment result in a second embodiment.

FIG. 16 is a diagram showing a functional configuration example in the second embodiment.

FIG. 17 is a diagram showing an example of heartbeat intervals and probability values in a third embodiment.

FIG. 18 is a diagram showing a first experiment result in the third embodiment.

FIG. 19 is a diagram showing a second experiment result in the third embodiment.

FIG. 20 is a diagram showing an example of a spectrogram and segment data in a fourth embodiment.

FIG. 21 is a diagram showing a first experiment result in a fourth embodiment.

FIG. 22 is a diagram showing a second experiment result in the fourth embodiment.

FIG. 23 is a diagram showing a third experiment result in the fourth embodiment.

FIG. 24 is a diagram showing a first experiment result obtained by calculating a heartrate interval in the fourth embodiment.

FIG. 25 is a diagram showing a second experiment result obtained by calculating a heartrate interval in the fourth embodiment.

FIG. 26 is a diagram showing a third experiment result obtained by calculating a heartrate in the fourth embodiment.

FIG. 27 is a diagram showing an example of IQ data measured by a Doppler radar.

### DESCRIPTION OF EMBODIMENTS

Optimum and minimum modes for carrying out the invention are explained below with reference to the drawings. Note that, in the drawings, when the same reference numerals and signs are added, the same reference numerals and signs indicate the same components. Redundant explanation of the components is omitted. Illustrated specific examples are illustrations. Components other than those illustrated may be further included.

### First Embodiment

For example, a biological detection system 1 is a system having an overall configuration explained below.

### Overall Configuration Example

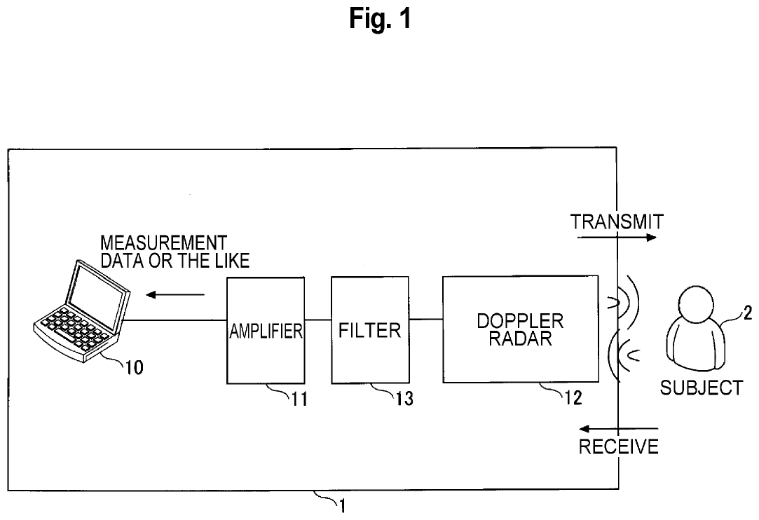

FIG. 1 is a diagram showing an overall configuration example in a first embodiment. For example, the biological detection system 1 has a configuration including a PC (Personal Computer, hereinafter referred to as “PC 10”), a Doppler radar 12, and a filter 13. Note that, as illustrated, the biological detection system 1 desirably includes an amplifier 11. In the following explanation, the illustrated overall configuration is explained as an example.

The PC 10 is an information processing device and is an example of a biological detection device. The PC 10 is connected to peripheral equipment such as the amplifier 11 via a network, a cable, or the like. Note that the amplifier 11 and the filter 13 may be components included in the PC 10. The amplifier 11, the filter 13, and the like may not be devices and may be configured by software or may be configured by both of hardware and software. In the following explanation, an illustrated example of the biological detection system 1 is explained.

The Doppler radar 12 is an example of a measurement device.

In this example, the PC 10 is connected to the amplifier 11. The amplifier 11 is connected to the filter 13. Further, the filter 13 is connected to the Doppler radar 12. The PC 10 acquires measurement data from the Doppler radar 12 via the amplifier 11 and the filter 13. That is, the measurement data is data of signals representing motions of an organism such as a heartbeat and respiration. Subsequently, the PC 10 analyzes body motions such as a heartbeat, respiration and a movement of the body of a subject 2 based on the acquired measurement data and measures a movement of a human body such as a heart rate.

The Doppler radar 12 acquires signals representing motions such as a heartbeat and respiration (hereinafter referred to as “biological signals”), for example, in a principle explained below.

### Example of the Doppler Radar

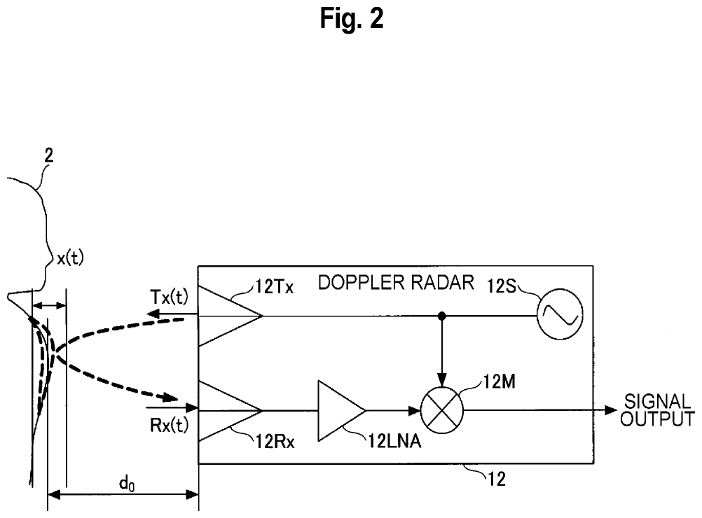

FIG. 2 is a diagram showing an example of the Doppler radar. For example, the Doppler radar 12 is a device having a configuration shown in FIG. 2. Specifically, the Doppler radar 12 includes a source 12S, a transmitter 12Tx, a receiver 12Rx, and a mixer 12M. The Doppler radar 12 includes an adjuster 12LNA such as an LNA (Low Noise Amplifier) that performs processing for, for example, reducing noise of data received by the receiver 12Rx.

The source 12S is a transmission source that generates a signal of a transmission wave transmitted by the transmitter 12Tx.

The transmitter 12Tx transmits a transmission wave to the subject 2. Note that a signal of the transmission wave can be shown by a function Tx(t) relating to a time “t” and, for example, can be shown by Expression (1) described below.

[Math. 1]

Tx(t)=cos(ω_{c}t) (Expression 1)

In Expression (1) described above, “ω_{c}” is an angular frequency of the transmission wave.

The subject 2, that is, a reflection surface of the transmitted signal is displaced by x(t) at the time “t”. In this example, the reflection surface is the chest wall of the subject 2. The displacement x(t) can be shown by Expression (2) described below.

[Math. 2]

x(t)=m×cos(ωt) (Expression 2)

In Expression (2) described above, “m” is a constant representing the amplitude of the displacement. In Expression (2) described above, “ω” is angular velocity shifted by a movement of the subject 2. Note that the variables common to Expression (1) described above are the same variables.

The receiver 12Rx receives a reflected wave that is a wave transmitted by the transmitter 12Tx and reflected by the subject 2. A signal of the reflected wave can be shown by a function Rx(t) relating to the time t and, for example, can be shown by Expression (3) described below.

$$\begin{matrix}[ Math . 3 ] & \\ Rx ( t ) = cos ( {ω}_{c} t - 2 π \cdot \frac{2 ( {d}_{0} + x ( t ) )}{λ} ) & ( Expression 3 )\end{matrix}$$

In Expression (3) described above, “d₀” is the distance between the subject 2 and the Doppler radar 12. “λ” is a wavelength of the signal. The distance and the wavelength are described the same below.

The Doppler radar 12 mixes the function Tx(t) (Expression (1) described above) representing the signal of the transmission wave and the function R(t) (Expression (3) described above) representing the signal of the reception wave and generates a Doppler signal. Note that, when the Doppler signal is shown by a function B(t) relating to the time t, the Doppler signal can be shown by Expression (4) described below.

$$\begin{matrix}[ Math . 4 ] & \\ B ( t ) = cos ( θ + 2 π \cdot \frac{2 x ( t )}{λ} ) & ( Expression 4 )\end{matrix}$$

When an angular frequency of the Doppler signal is represented as “ω_{d}”, the angular frequency ω_{d} of the Doppler signal can be shown by Expression (5) described below.

$$\begin{matrix}[ Math . 5 ] & \\ {ω}_{d} = θ + 2 π \cdot \frac{2 x ( t )}{λ} & ( Expression 5 )\end{matrix}$$

A phase “θ” in Expression (4) described above and Expression (5) described above can be shown by Expression (6) described below.

$$\begin{matrix}[ Math . 6 ] & \\ θ = 2 π \cdot \frac{2 {d}_{0}}{λ} + {θ}_{0} & ( Expression 6 )\end{matrix}$$

In Expression (6) described above, “θ₀” is phase displacement on the chest wall of the subject 2, that is, the reflection surface.

Subsequently, the Doppler radar 12 outputs the position, the speed, and the like of the subject 2 based on a result of comparing the signal of the transmitted transmission wave and the signal of the received reception wave, that is, calculation results of the expressions described above.

For example, I data (in-phase data) and Q data (orthogonal phase data) can be generated from the reception wave. A distance that the chest wall of the subject 2 moved can be detected according to the I data and the Q data. It is possible to detect, based on phases shown by the I data and the Q data, in which of the front and the rear the chest wall of the subject 2 moved. Therefore, an indicator such as a heartbeat can be detected from a movement of the chest wall deriving from a heartbeat using frequency changes of the transmission wave and the reception wave.

### Hardware Configuration Example of the Biological Detection Device

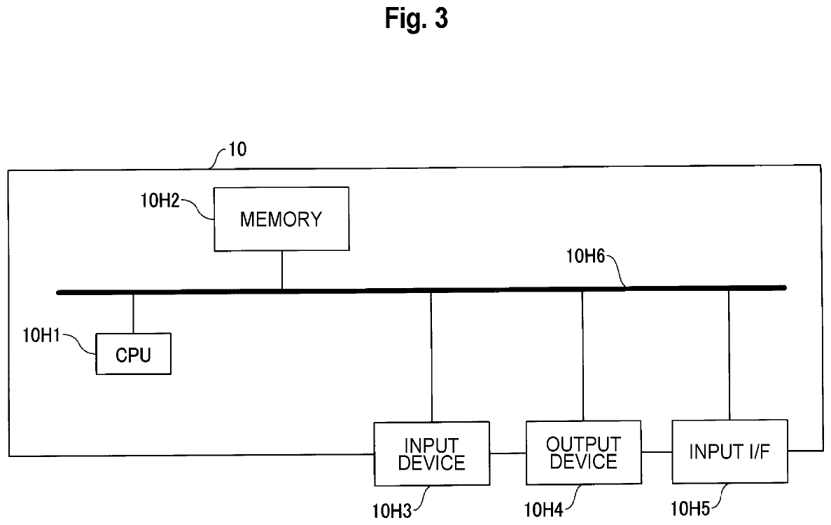

FIG. 3 is a diagram showing an example of the biological detection device. For example, the PC 10 includes a CPU (Central Processing Unit, hereinafter referred to as “CPU 10H1”), a memory 10H2, an input device 10H3, an output device 10H4, and an input I/F (Interface) (hereinafter referred to as “input I/F 10H5”). Note that the respective pieces of hardware included in the PC 10 are connected by a bus (hereinafter referred to as “bus 10H6”). Data and the like are mutually transmitted and received among the respective pieces of hardware via the bus 10H6.

The CPU 10H1 is a control device that controls the hardware included in the PC 10 and an arithmetic device that performs an arithmetic operation for realizing various kinds of processing.

The memory 10H2 is, for example, a main memory and an auxiliary memory. Specifically, the main memory is, for example, a memory. The auxiliary memory is, for example, a hard disk. The memory 10H2 stores data including intermediate data used by the PC 10, programs used for the various kinds of processing and the control, and the like.

The input device 10H3 is a device for inputting parameters and instructions necessary for calculation to the PC 10 according to operation of a user. Specifically, the input device 10H3 is, for example, a keyboard, a mouse, and a driver.

The output device 10H4 is a device for outputting various processing results and calculation results by the PC 10 to the user and the like. Specifically, the output device 10H4 is, for example, a display.

The input I/F 10H5 is an interface connected to an external device such as a measurement device to transmit and receive data and the like. For example, the input I/F 10H5 is a connector, an antenna, or the like. That is, the input I/F 10H5 transmits and receives data to and from the external device via a network, radio, or a cable.

Note that a hardware configuration is not limited to the illustrated configuration. For example, the PC 10 may further include an arithmetic device or a memory for performing processing in parallel, decentrally, or redundantly. The PC 10 may be an information processing system connected to other devices via a network or a cable in order to perform an arithmetic operation, control, and storage in parallel, decentrally, or redundantly. That is, the present invention may be realized by an information processing system including one or more information processing devices.

In this way, the PC 10 acquires a biological signal representing a motion of an organism with the measurement device such as the Doppler radar 12. Note that the biological signal may be acquired at any time in real time or a device such as the Doppler radar may store biological signals for a certain period and, thereafter, the PC 10 may collectively acquire the biological signals. A recording medium or the like may be used for the acquisition. Further, the PC 10 may include the measurement device such as the Doppler radar 12 and the PC 10 may perform measurement with the measurement device such as the Doppler radar 12, generate a biological signal, and acquire the biological signal.

### Overall Processing Example

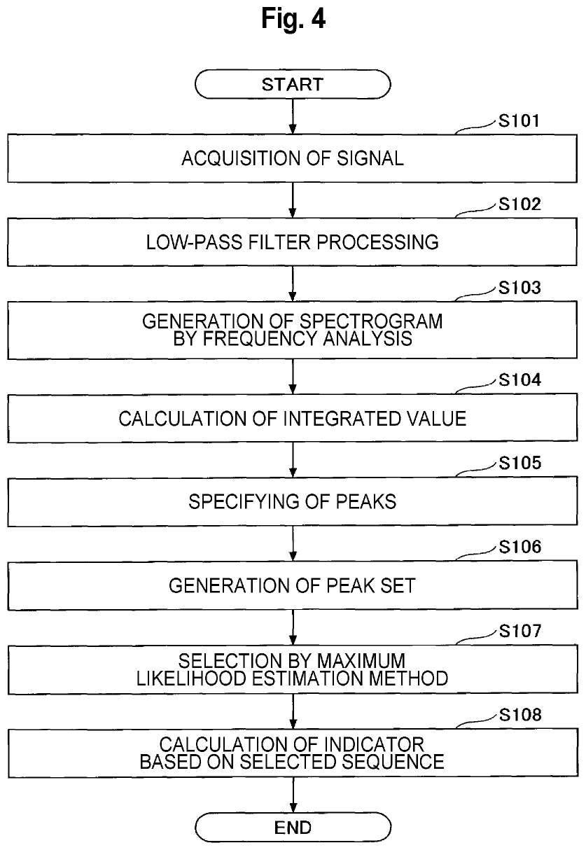

FIG. 4 is a diagram showing an overall processing example. For example, overall processing explained below is repeatedly executed for each predetermined time.

### Acquisition Example of a Signal

In step S101, the PC 10 acquires a signal generated by measuring an organism with a Doppler radar or the like, the signal representing a measurement result including a heartbeat component, (hereinafter simply referred to as “signal”). For example, the signal is a signal explained below.

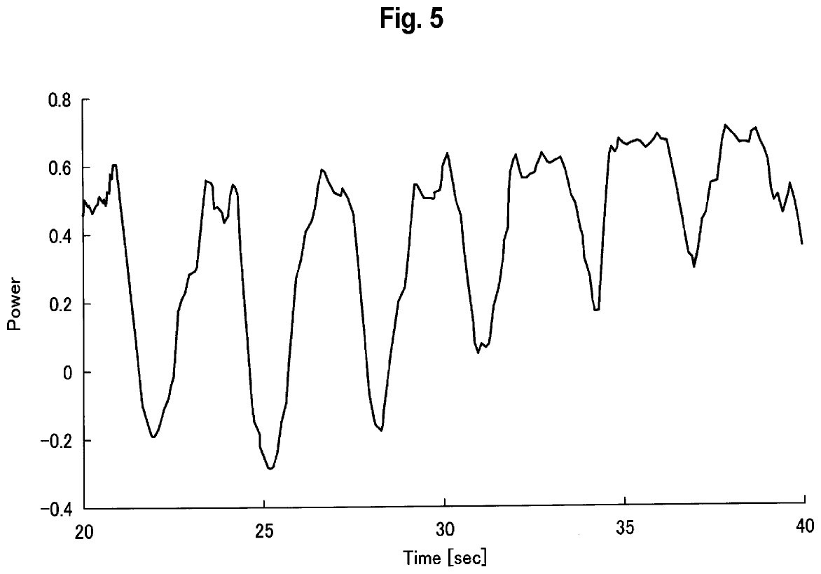

FIG. 5 is a diagram showing an example of a first signal. In the figure, the horizontal axis represents a time showing a point in time when measurement is performed. On the other hand, the vertical axis represents electric power estimated based on a measurement result of the Doppler radar.

### Example of Low-Pass Filter Processing

In step S102, the PC 10 performs, on a signal, low-pass filter processing for attenuating a frequency component higher than a frequency component that a heartbeat generally takes. Such preprocessing may be performed on the signal.

Specifically, the PC 10 performs, on the signal, filter processing for attenuating a frequency component higher than the frequency component of the heartbeat. For example, the PC 10 performs, with a digital filter or the like, filtering using a frequency higher than the frequency component of the heartbeat set as a cutoff frequency.

For example, a male adult has a heart rate of approximately 50 to 180 per one minute. Therefore, a frequency component of a heartbeat, that is, a frequency component of a heartbeat is mainly a frequency component of approximately 0.8 Hz to 3 Hz.

Therefore, the low-pass filter processing is desirably set to attenuate, for example, a frequency component higher than 0 Hz to 3 Hz. With such setting, the PC 10 can attenuate a frequency component, which is noise, without attenuating frequency components representing respiration and a heartbeat with the low-pass filter processing.

In this way, the PC 10 desirably performs the preprocessing such as the low-pass filter processing not to attenuate a frequency band including the heartbeat but to attenuate a frequency component higher than the frequency component of the heartbeat.

Note that a frequency band targeted by the low-pass filter processing may be set considering age, sex, a state, and the like of an organism. For example, a state after vigorous exercise or in an excited state, a heart rate has a frequency higher than a frequency in a quiet state. Therefore, in both the cases, a frequency of the heartbeat is a frequency higher than the frequency in the quiet states. On the other hand, in the quiet state, the frequency of the heartbeat is a low frequency.

Therefore, for example, the frequency band targeted by the low-pass filter processing is dynamically changed according to the state or the like or the frequency band targeted by the low-pass filter processing may be narrowed.

Specifically, in a state considered to be in a frequency band in which the frequency of the heartbeat is high such as the state after a vigorous exercise, low-pass filter processing for attenuating a frequency component higher than 3.5 Hz is performed. On the other hand, in a state in which the frequency of the heartbeat is considered to be in a low frequency band such as the quiet state, low-pass filter processing for attenuating a frequency component higher than 1.4 Hz is performed.

In this way, the state or the like can be input or a value considering the state or the like may be set and the low-pass filter processing may be performed.

For example, in low-pass filter processing set to 3 Hz, the PC 10 does not attenuate a frequency component of a heartbeat that occurs after vigorous exercise in which the heartbeat is approximately 180 per one minute and can attenuate a frequency component of noise. Therefore, when it is known that a state is not after an organism exercises, a value as low as 1 Hz may be set for the low-pass filter processing.

The signal may be a signal subjected to such preprocessing.

### Generation Example of a Spectrogram by a Frequency Analysis

In step S103, the PC 10 performs a frequency analysis of the signal to generate a spectrogram. For example, the frequency analysis is realized by FFT (Fast Fourier Transform) or the like. In this way, the PC 10 calculates a spectrum showing energy for each frequency band. The PC 10 may perform normalization to generate a spectrogram.

For example, a spectrogram is generated as explained below.

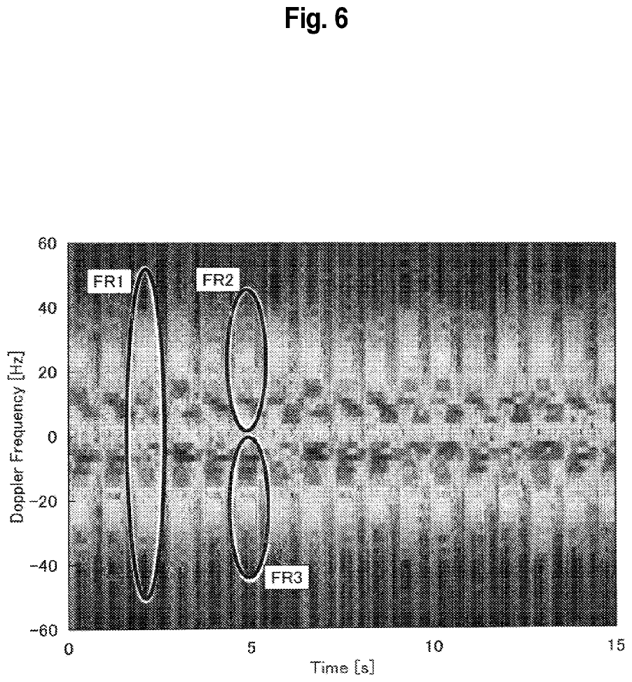

FIG. 6 is a diagram showing an example of a spectrogram. In the following explanation, a measurement result in a state in which the subject 2 is seated and stands still is explained as an example.

The spectrogram includes a frequency component corresponding to a heartbeat (in the figure, indicated by “heartbeat frequency component FR1”). The heartbeat frequency component FR1 includes a component belonging to a positive frequency domain (in the figure, indicated by “positive frequency component FR2”) and a component belonging to a negative frequency domain (in the figure indicated by “negative frequency component FR3”).

Specifically, the positive frequency component FR2 is a frequency component of a frequency (in the figure, indicated by the vertical axis) equal to or higher than “0”. On the other hand, the negative frequency component FR3 is a frequency component of a frequency lower than “0”.

The positive frequency component FR2 is a frequency component due to expansion of a heart. The positive frequency component FR2 is a spectrum centering on a frequency of 8 Hz to 30 Hz. On the other hand, the negative frequency component FR3 is a frequency component due to contraction of the heart. The negative frequency component FR3 is a spectrum centering on a frequency of −8 Hz to −30 Hz.

### Calculation Example of an Integrated Value

In step S104, the PC 10 calculates an integrated value. Specifically, the integrated value is calculated by integrating (discretely, adding up) power (in the figure, the intensity of the power is indicated by light and shade) of the heartbeat frequency component FR1 among frequency components shown by the spectrogram. In the following explanation, an example is explained in which energy is the power. That is, the energy is calculated from a change in a voltage (or an electric current) on a lead wire by the PC 10 after energy of an electromagnetic wave is guided to the lead wire by an antenna. In this way, the energy only has to be a value representing energy at each of times and frequencies. An acquisition method and a calculation method for the energy do not matter.

In the example of the spectrogram shown in FIG. 6, the integrated value is calculated, for example, as explained below.

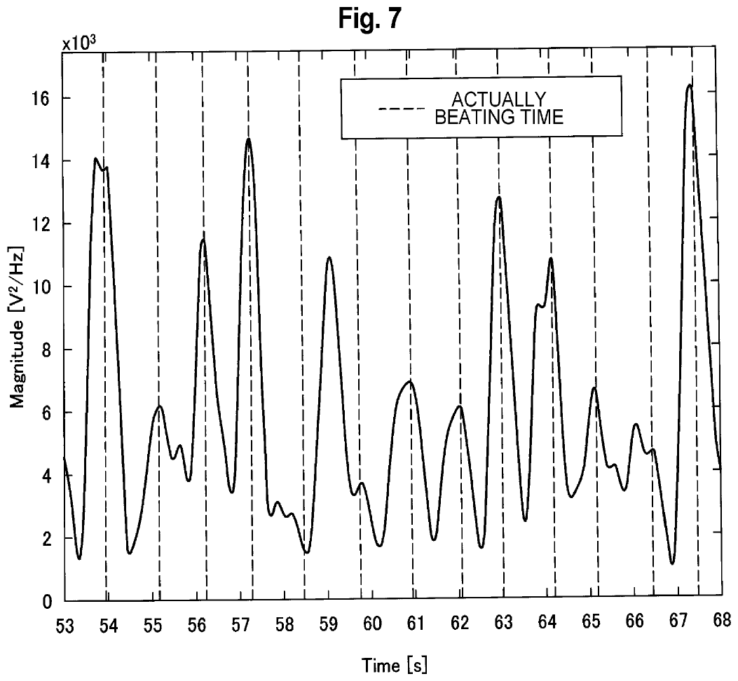

FIG. 7 is a diagram showing a calculation result example of the integrated value. Specifically, the integrated value is calculated by integrating spectra generated at the heartbeat frequency component FR1, that is, 8 Hz to 30 Hz and −8 Hz to −30 Hz.

### Peak Specifying Example

In step S105, the PC 10 specifies peaks. For example, the peaks are maximum values at the integrated value. Therefore, the integrated value is differentiated and the peaks are specified by, for example, points where differentiated values are “0”. Note that the peaks are not limited to be specified by the method of using the maximum values but may be specified by another method. For example, the peaks are specified as explained below.

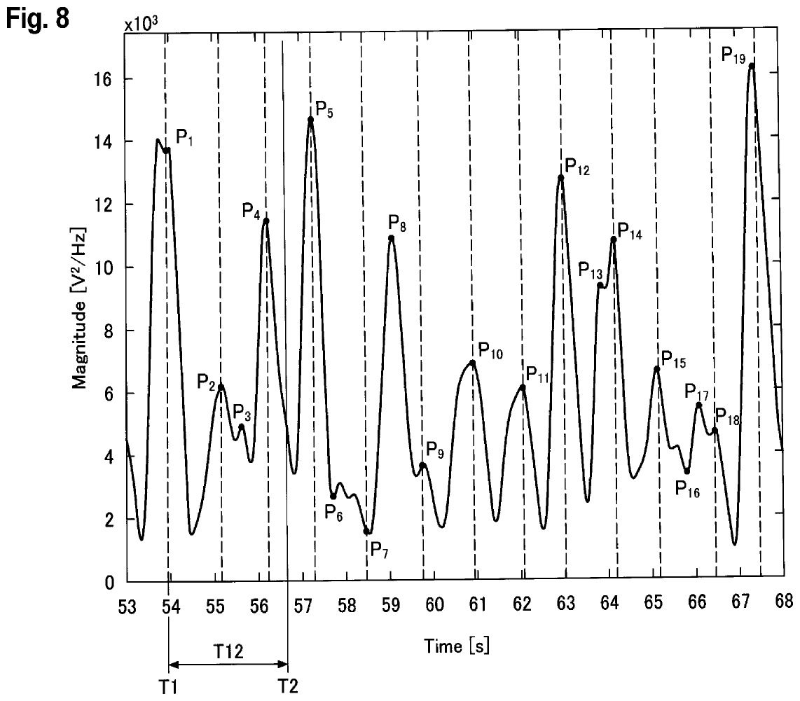

FIG. 8 is a diagram showing a peak specifying example. For example, peaks are points indicated by “Pn” (n=1, 2, 3, . . . ) in the figure. Note that “n” is a number representing occurrence order in a time and is a value numbered in time order for the detected peaks. In the following explanation, as shown in FIG. 8, an example is explained in which nineteen peak candidates “P1” to “P19” are detected.

### Peak Set Generation Example

In step S106, the PC 10 generates a peak set.

The peak set is a combination of peaks. In the following explanation, one peak of peaks configuring the peak set is referred to as “first peak”. Of two peaks including the first peak configuring the peak set, a peak generated in a time later than the first peak is referred to as “second peak”.

In the following explanation, an example is explained in which “P₁” in FIG. 8 is set as the first peak, the second peak paired with the first peak is selected, and the peak set is generated.

The second peak is a peak included in a time that a heartbeat can take (hereinafter referred to as “second peak occurrence time T2”) based on a time when the first peak occurs (hereinafter referred to as “first peak occurrence time T1”). Specifically, a time from the first peak occurrence time T1 to the second peak occurrence time T2 (hereinafter referred to as “peak detection time T12”) is often approximately 2 seconds. However, there is an individual difference or the like in the peak detection time T12. Therefore, considering the individual difference or the like, the peak detection time T12 is desirably set to approximately 2.5 seconds. Note that the peak detection time T12 is set beforehand. The peak detection time T12 may be set considering a state or the like of an organism.

That is, a peak that occurs within 2.5 seconds from occurrence of “P₁” is specified as the second peak. Specifically, the example shown in FIG. 8 is an example in which “P₂” to “P₄” occur within 2.5 seconds from the first peak occurrence time T1 to the second peak occurrence time T2. Accordingly, three peaks “P₂” to “P₄” are specified as the second peaks for the first peak “P₁”.

Three peak sets including “P₁” as the first peak and including “P₂” to “P₄” as the second peaks are generated. When a plurality of peaks are specified in the peak detection time T12 in this way, a plurality of peak sets are generated by pairing the respective second peaks and the first peak.

The PC 10 generates the next peak set in the same manner. For example, when all peak sets including “P₁” as the first peak are generated, “P₂” that occurs next to “P₁” is set as the first peak and the second peak is specified in the same manner. In this way, peak sets are generated for all specified peaks.

### Selection Example By a Maximum Likelihood Estimation Method

In step S107, the PC 10 selects, with a maximum likelihood estimation method, a most likely sequence in the maximum likelihood estimation method from the peak set. Note that the maximum likelihood estimation method is desirably a Viterbi algorithm. However, the maximum likelihood estimation method may be a method other than the Viterbi algorithm and only has to be a method that can calculate likelihood. For example, the maximum likelihood estimation method may be a method of performing full search. Note that, when the Viterbi algorithm is used, a heartbeat can be efficiently detected. In the following explanation, an example in which the Viterbi algorithm is used is explained.

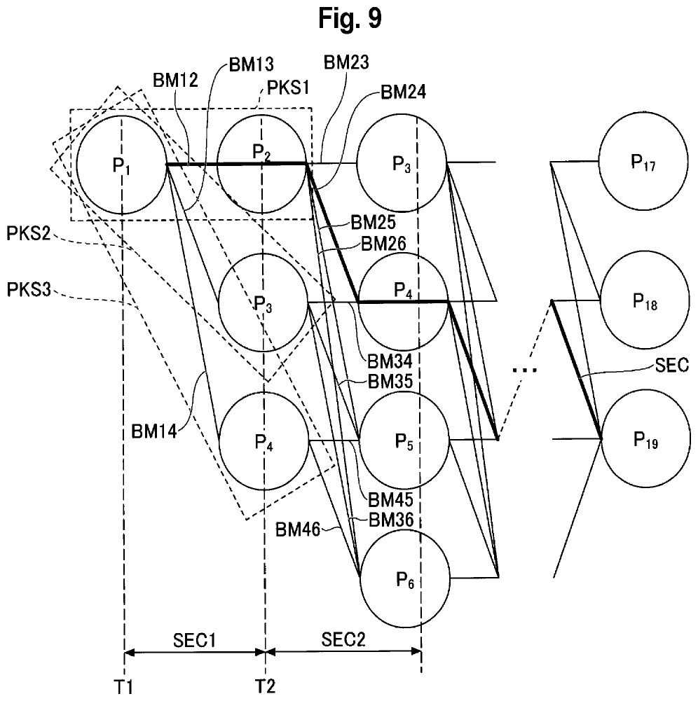

FIG. 9 is a diagram showing an example of selection by the maximum likelihood estimation method. In the following explanation, three peak sets including “P₁” as the first peak are explained as an example. In the following explanation, an example is explained in which a first peak set PKS1 to a third peak set PKS3, which are the three peak sets including “P₁” as the first peak and including “P₂” to “P₄” as the second peaks, are generated by step S106.

The first peak set PKS1 is a peak set that is a combination of the first peak “P₁” and the second peak

The second peak set PKS2 is a peak set that is a combination of the first peak “P₁” and the second peak “P₃”.

The third peak set PKS3 is a peak set that is a combination of the first peak “P₁” and the second peak

“P₄”.

In the figure, likelihood between the first peak and the second peak configuring the peak set (hereinafter referred to as “peak-to-peak value”) is indicated by a line connecting a peak and a peak. In the following explanation, an example is explained in which the peak-to-peak value is a “branch metric” in the Viterbi algorithm. Note that the peak-to-peak value only has to be a calculation result representing likelihood. A calculation method for the peak-to-peak value is explained below.

In the following explanation, a branch metric of “P₁” and “P₂” is referred to as “twelfth branch metric BM12”. Similarly, a branch metric of “P₁” and “P₃” is referred to as “thirteenth branch metric BM13” and a branch metric of “P₁” and “P₄” is referred to as “fourteenth branch metric BM14”. Branch metrics are calculated in the same manner for the third peak set PKS3 and the subsequent peak sets (in the figure, peak sets in which peaks described further on the right side than “P₂” to “P₄” are used) as “twenty-third branch metric BM23”, “twenty-fourth branch metric BM24”, “twenty-fifth branch metric BM25”, “thirty-fourth branch metric BM34” . . . .

From the Viterbi algorithm, the PC 10 selects a sequence having the highest likelihood among the combinations of the peak sets. Specifically, the example shown in FIG. 9 is an example in which a sequence SEC is selected by the algorithm.

A value obtained by accumulating branch metrics for each of sections (hereinafter referred to as “cumulative value”) is calculated. In the following explanation, an example is explained in which the cumulative value is a “path metric” in the Viterbi algorism. As illustrated, the sections are divided like a “first section SEC1”, a “second section SEC2”, . . . according to a time sequence. Then, branch metrics are selected one by one for each of the sections. A path metric can be selected by connecting the branch metrics.

That is, the sequence SEC having the highest likelihood is a sequence in which the path metric is the minimum or the maximum (by a method of calculating likelihood of the minimum or the maximum).

In this example, first, among the twelfth branch metric BM12, the thirteenth branch metric BM13, and the fourteenth branch metric BM14, the twelfth branch metric BM12 is determined as having the highest likelihood.

Subsequently, when the twelfth branch metric BM12 is selected, branch metrics are selected in the same manner for a peak set including “P₂” as the first peak and including “P₃” to “P₆” as the second peaks. In this example, it is assumed that the twenty-fourth branch metric BM24 is determined as having the highest likelihood.

When branch metrics, that is, peak sets are selected up to “P₁₉” in this way, one sequence SEC connecting the peaks of “P₁” to “P₁₉” is selected. When the sequence SEC is selected in this way, a combination of peaks representing a heartbeat can be extracted. That is, the sequence SEC shows a result obtained by extracting peaks representing the heartbeat among specified peaks. If such a sequence SEC can be selected, the heartbeat can be accurately measured.

### Calculation Example of the Peak-To-Peak Value by a Heartbeat Interval

In the following explanation, an example is explained in which a heartbeat interval is an RRI (R-R interval).

A signal representing a heartbeat includes crests (equivalent to maximum points) or valleys (equivalent to minimum points) of a signal called “P”, “Q”, “R”, “S”, and “T” generated by electricity caused in the heart by contraction and extraction of the heart. The RRI is an interval from a point in time at a peak such as “R” to a point in time at the next “R”. That is, the heartbeat interval is a value representing a time of one beat in the heartbeat. Therefore, the heartbeat interval may be a value other than the RRI. In the following explanation, the RRI is explained as an example. However, since “R” is often clearer than the other crests or valleys in the heartbeat, the heartbeat can be accurately measures if the RRI is used.

The RRI represents an interval between a peak and a peak. That is, the RRI is calculated by specifying a reference peak and the next peak and measuring a time between the peaks.

In the following explanation, an example is explained in which “R” is equivalent to peaks that occur in the order of a “first point in time TR1”, a “second point in time TR2, a “third point in time TR3”, . . . .

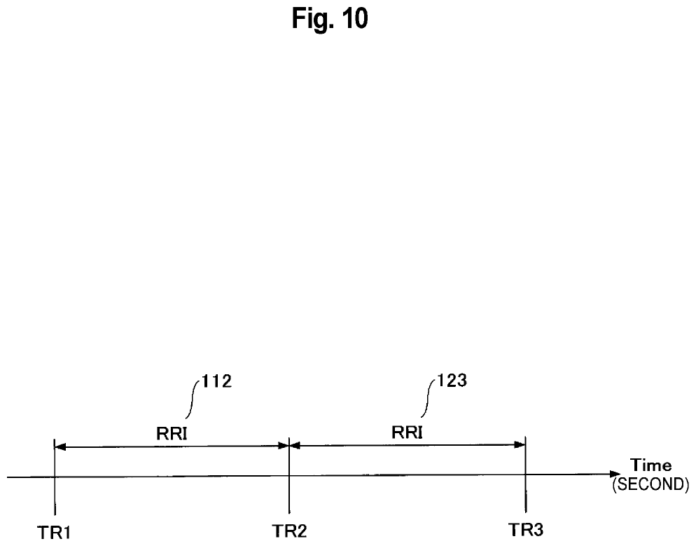

FIG. 10 is a diagram showing an example of a heartbeat interval. For example, as illustrated, when peaks occur at respective points in time of the “first point in time TR1”, the “second point in time TR2”, the “third point in time TR3”, . . . , an interval between the first point in time TR1 and the second point in time TR2 is an RRI (hereinafter referred to as “twelfth RRI112”). On the other hand, the next RRI of the twelfth RRI112, that is, an interval between the second point in time TR2 and the third point in time TR3 is an RRI (hereinafter referred to as “twenty-third RRI123”).

In the following explanation, the twelfth RRI112 is explained as an example of a first heartbeat interval and the twenty-third RRI123 is explained as an example of a second heartbeat interval. A relation between preceding and following RRIs in a time axis like the twelfth RRI112 and the twenty-third RRI123 is sometimes expressed as “adjacent”.

A branch metric is calculated by squaring the difference between adjacent RRIs considering a case in which the difference is a positive value and a negative value. Specifically, Expression (8) described below and Expression (11) described below are substituted in Expression (10) described below. A calculation result of Expression (12) described below is obtained by such calculation. As shown by Expression (12) described below, a square of the difference between RRIs remains in the calculation result, that is, Expression (12) described below. Therefore, the square of the difference between the RRIs obtained in this way is adopted as the branch metric.

If the difference between the adjacent RRIs, that is, the twelfth RRI112 and the twenty-third RRI123 is due to the peaks of the heartbeat, an average value occurs according to a normal distribution (called “Gaussian distribution” as well) of “0”. Accordingly, if the difference between the adjacent RRIs is represented as a graph, for example, the difference has a tendency explained below.

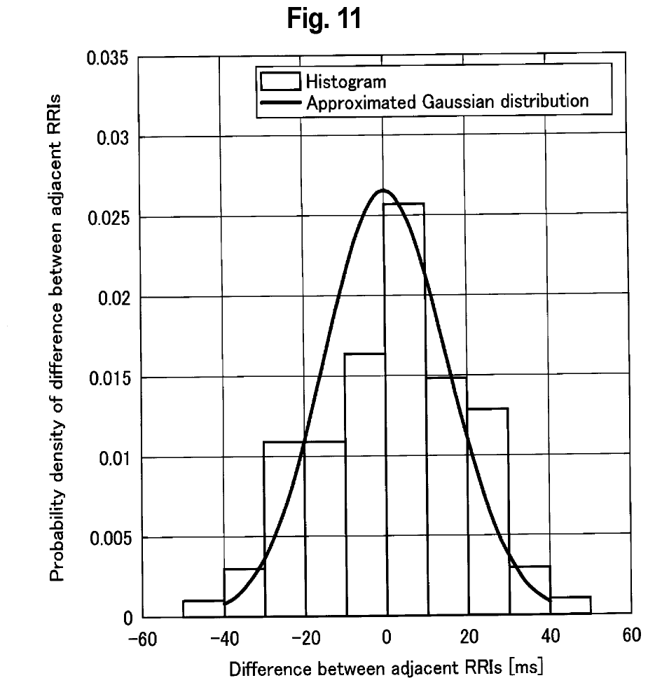

FIG. 11 is a diagram showing an example of a histogram of the difference between the adjacent RRIs. Specifically, the difference between the adjacent RRIs like the twelfth RRI112 and the twenty-third RRI123 has an average value of “0” (“0” of the horizontal axis in the figure) and is in a relation of the normal distribution. That is, if the difference between two RRI is in the relation of the normal distribution, it is highly likely that peaks representing a heartbeat are detected.

Note that the normal distribution has a tendency or a deviation of dispersion depending on a person or a state. That is, a kurtosis, a skewness, or both may be set in the normal distribution for each person or state.

Therefore, if the difference between the adjacent RRIs is represented as “Sᵢ”, Expression (7) described below and Expression (8) described below hold. Note that, in Expression (7) described below and Expression (8) described below, “i” is a peak number representing appearance order given to peaks in time order.

$$\begin{matrix}[ Math . 7 ] & \\ {S}_{i} = \vert {RRI}_{i} + {RRI}_{i + 1} \vert & ( Expression 7 )\end{matrix}$$

$$\begin{matrix}[ Math . 8 ] & \\ P ( {S}_{i} ) = \frac{1}{\sqrt{2 π {δ}^{2}}} exp \{ - ( {S}_{i}^{2} / 2 {δ}^{2} ) \} & ( Expression 8 )\end{matrix}$$

δ2: Dispersion of the difference between the adjacent RRIs

The differences between the respective adjacent RRIs, that is, the differences between the RRIs are calculated for each of peak sets. A set of the differences between the adjacent RRIs calculated in this way (hereinafter simply referred to as “set”) is represented as “X”. That is, the set “X” is a set of the differences between the adjacent RRIs as shown by Expression (9) described below. Note that “M” in Expression (9) described below is a serial number of the difference between the adjacent RRIs decided by the number of peak sets.

[Math. 9]

X={S₁, S₂, S₃ . . . S_{M}} (Expression 9)

In step S107, a set that is a combination of differences “Sᵢ” of the most likely adjacent RRIs is selected as a sequence in Expression (9) described above. When “X” selected in this way is represented as a selected sequence set “Xₛ”, a sequence is selected in step S107 according to the selected sequence set “Xₛ” calculated as shown by Expression (10) described below.

$$\begin{matrix}[ Math . 10 ] & \\ {X}_{S} = {argmax}_{X} P ( X ) & ( Expression 10 )\end{matrix}$$

A function “P(X)” in Expression (10) described above is a function shown in Expression (11) described below.

$$\begin{matrix}[ Math . 11 ] & \\ P ( X ) = {\prod }_{i = 1}^{M} P ( {S}_{i} ) & ( Expression 11 )\end{matrix}$$

A relation shown by Expression (12) described below holds according to Expression (7) described above, Expression (8) described above, and Expression (10) described above.

$$\begin{matrix}[ Mat . 12 ] & \\ {X}_{S} = {argmax}_{X} {\sum }_{i = 1}^{M} - {S}_{i}^{2} = {argmin}_{X} {\sum }_{i = 1}^{M} {S}_{i}^{2} & ( Expression 12 )\end{matrix}$$

As shown by Expression (12) described above, the selected sequence set “Xₛ” is decided by a value obtained by squaring the difference between the adjacent RRIs. Accordingly, if a sequence in which a total is the minimum is selected using the value obtained by squaring the difference between the adjacent RRIs as the branch metric, a sequence having the highest likelihood can be selected.

Note that the likelihood can be calculated by a humming distance or the like. However, in the method of calculating the likelihood based on the difference between the adjacent RRIs, the likelihood can be calculated without substitution or the like in calculating the likelihood.

### Example in Which Calculation of an Indicator is Performed

In step S108, the PC 10 may calculate an indicator based on the selected sequence.

For example, the indicator is a value representing biological information of a target organism. Specifically, the indicator is a value calculated by analyzing a biological signal and is a pulse rate, a heart rate, a respiration rate, a blood pressure, a PTT (pulse transit time), a systolic blood pressure, an RRI, a QRS interval, a QT interval, a combination of the foregoing, or the like. Note that the indicator may be biological information other than the above.

### Functional Configuration Example

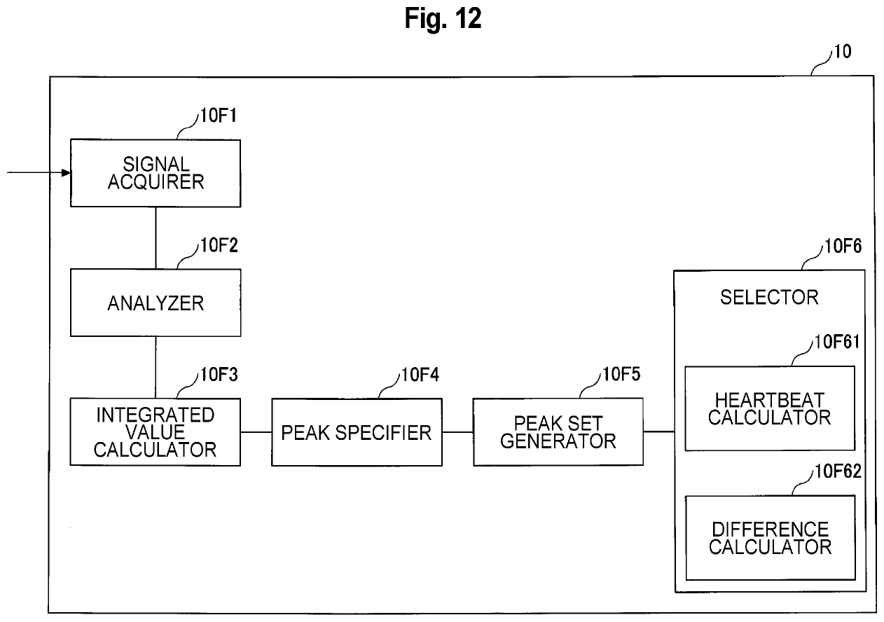

FIG. 12 is a diagram showing a functional configuration example in the first embodiment. For example, the PC 10 is a functional component including a signal acquirer 10F1, an analyzer 10F2, an integrated value calculator 10F3, a peak specifier 10F4, a peak set generator 10F5, and a selector 10F6.

The signal acquirer 10F1 performs a signal acquisition procedure for acquiring a signal including a heartbeat. For example, the signal acquirer 10F1 is realized by the Doppler radar 12, the input I/F 10H5, or the like.

The analyzer 10F2 performs a frequency analysis procedure for performing a frequency analysis of a signal and generating a spectrogram. For example, the analyzer 10F2 is realized by the CPU 10H1 or the like.

The integrated value calculator 10F3 performs an integrated value calculation procedure for integrating energy of a heartbeat frequency component, which is a frequency component corresponding to a heartbeat, among frequency components shown by the spectrogram generated by the analyzer 10F2 and calculating an integrated value. For example, the integrated value calculator 10F3 is realized by the CPU 10H1 or the like.

The peak specifier 10F4 performs a peak specifying procedure for specifying peaks in the integrated value. For example, the peak specifier 10F4 is realized by the CPU 10H1 or the like.

The peak set generator 10F5 performs a peak set generation procedure for combining a first peak and a second peak among the peaks specified by the peak specifier 10F4 and generating a peak set. For example, the peak set generator 10F5 is realized by the CPU 10H1 or the like.

The selector 10F6 performs a selection procedure for selecting from the peak set generated by the peak set generator 10F5, a sequence having the highest likelihood in the maximum likelihood estimation method. For example, the selector 10F6 is realized by the CPU 10H1 or the like.

For example, the selector 10F6 has a configuration including a heartbeat interval calculator 10F61 and a difference calculator 10F62.

The heartbeat interval calculator 10F61 calculates heartbeat intervals such as a first heartbeat interval and a second heartbeat interval.

The difference calculator 10F62 calculates the difference between adjacent heartbeat intervals among the heartbeat intervals calculated by the heartbeat interval calculator 10F61.

The maximum likelihood estimation method in which, for example, a value obtained by squaring the difference calculated by the difference calculator 10F62 is used as a peak-to-peak value is performed.

### Second Embodiment

Compared with the first embodiment, a second embodiment is different in a calculation method for likelihood. In the following explanation, differences from the first embodiment are mainly explained and redundant explanation of the explanation in the first embodiment is omitted.

A spectrogram is generated by, for example, the method explained in the first embodiment. In the following explanation, an example is explained in which the spectrogram is generated by the method explained in the first embodiment.

In the second embodiment, in calculating likelihood, the spectrogram is divided as explained below to generate segment data.

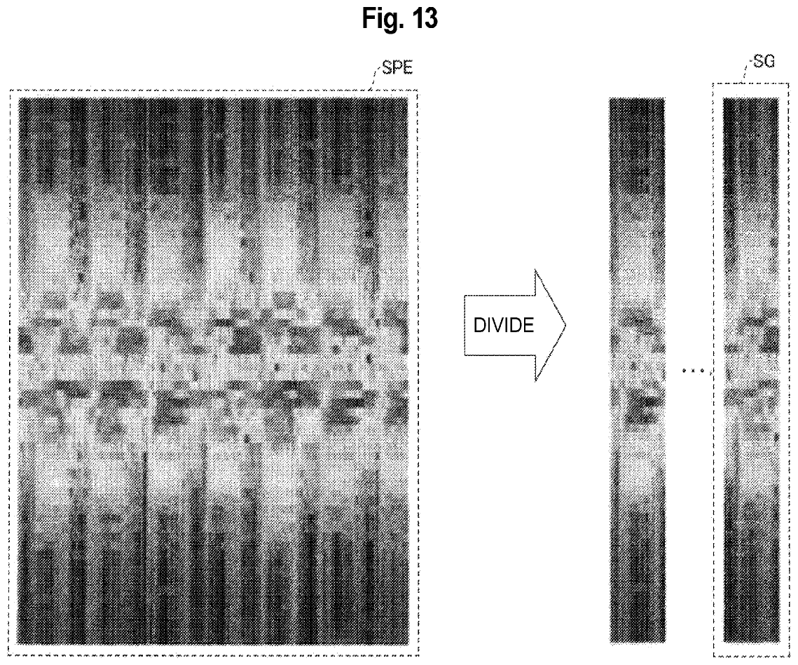

FIG. 13 is a diagram showing a generation example of segment data. In the following explanation, an example is explained in which a spectrogram SPE shown in FIG. 13 is generated. When the spectrogram SPE is divided in predetermined time units (in the figure, at equal intervals in the horizontal axis direction), a plurality of segment data SG can be generated.

The segment data SG generated in this way are input to a learned model learned beforehand. A probability value of the respective segment data SG being data representing a heartbeat is output as explained below.

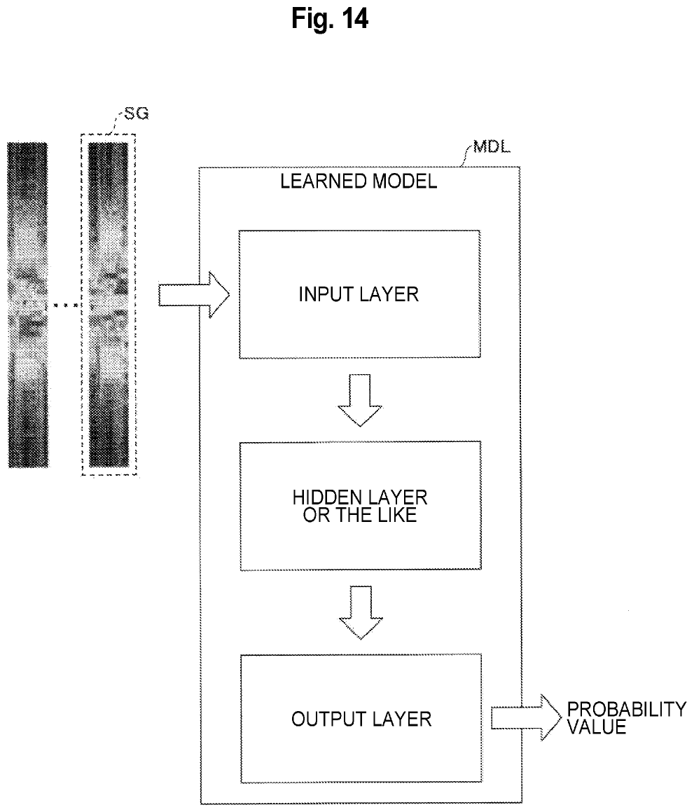

FIG. 14 is a diagram showing an output example of the probability value by the learned model. For example, a learned model MDL has a network structure such as a CNN (Convolution Neural Network). Note that the learned model MDL may be realized by other machine learning.

For example, the learned model MDL is generated by performing machine learning with teacher data, which is a set of the segment data SG and a result of determining whether the segment data SG represent a heartbeat. That is, the learned model MDL is a learned model or the like generated by a supervised scheme. With such a configuration, for example, it is possible to accurately determine a probability value of being a heartbeat as explained below.

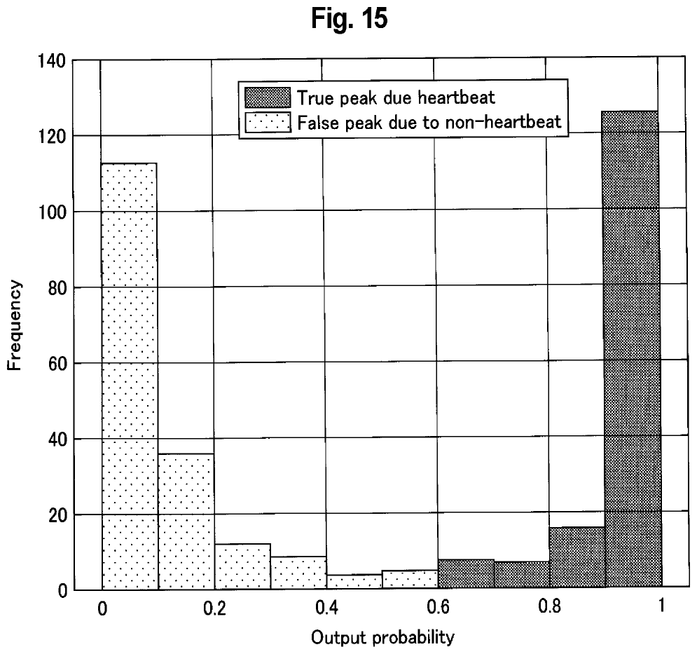

FIG. 15 is a diagram illustrating an experiment result in the second embodiment. In the figure, a probability value output by an experiment is shown on the horizontal axis. The vertical axis represents the number of times. “True peak due heartbeat” indicates an experiment result for segment data generated from a spectrogram obtained by actually measuring a heartbeat. As illustrated, a probability value with a high value (in the figure, a value close to “1”) was successfully output with respect to the “True peak due heartbeat”.

On the other hand, “False peak due heartbeat” indicates an experiment result for segment data generated from a spectrogram that is not a heartbeat. As illustrated, a probability value with a low value (in the figure, a value close to “0”) was successfully output with respect to the “False peak due heartbeat”.

When the learned model is used in this way, it is possible to accurately output a probability value, which is data representing a heartbeat, with respect to an input of segment data. Accordingly, when a sequence in which a total is maximized is selected using a probability value as the branch metric or the like, it is possible to select a sequence having the highest likelihood.

### Functional Configuration Example

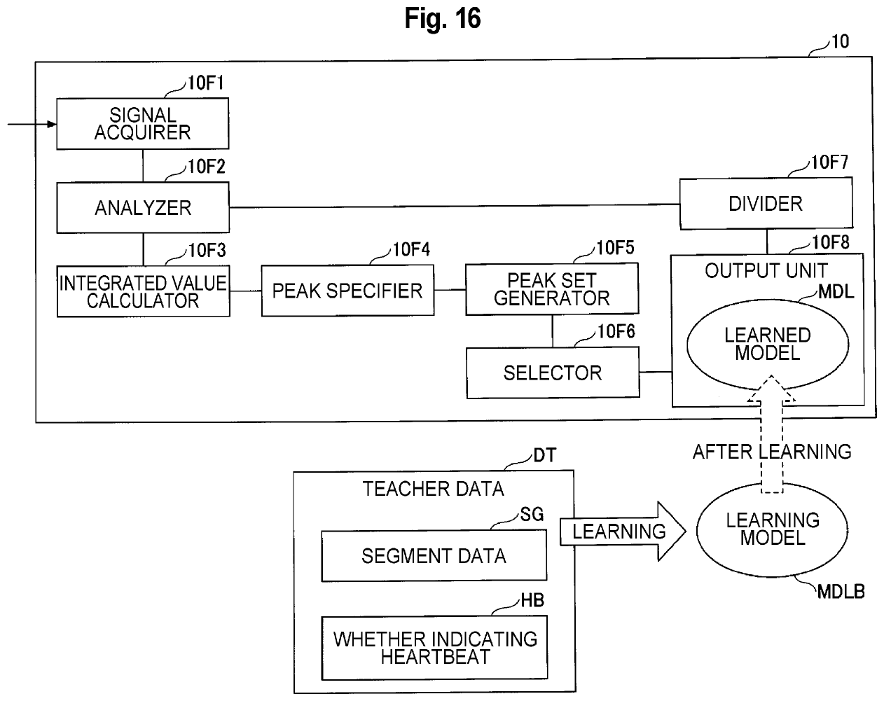

FIG. 16 is a diagram showing a functional configuration example in the second embodiment. For example, compared with the first embodiment, the PC 10 is the same in a configuration including the signal acquirer 10F1 and the analyzer 10F2.

On the other hand, the second embodiment is different in that the PC 10 has a functional configuration including a divider 10F7 and an output unit 10F8.

The divider 10F7 generates segment data obtained by dividing a spectrogram in predetermined time units.

The output unit 10F8 outputs, with the learned model MDL, a probability value of the segment data being data representing a heartbeat.

When the probability value output by the output unit 10F8 is used as the peak-to-peak value in the first embodiment, the selector 10F6 can select a sequence having the highest likelihood in the maximum likelihood estimation method and accurately measure a heart rate. For example, as in the first embodiment, a peak set is generated by a configuration including the integrated value calculator 10F3, the peak specifier 10F4, and the peak set generator 10F5.

The learned model MDL is generated by causing the PC 10 to learn a learning model MDLB using teacher data DT that is a set of the segment data SG and correct answer data HB about whether the segment data SG represents a heartbeat. Note that the learning of the learning model MDLB may be performed by another device or may be performed by the PC 10. Alternatively, the learning of the learning model MDLB may be additionally performed by the PC 10 after being performed by the other device.

### Third Embodiment

A third embodiment is an embodiment in which the branch metric calculated by the method explained in the first embodiment and the branch metric calculated by the method explained in the second embodiment are combined.

Specifically, in the third embodiment, a branch metric “P′(Sᵢ)” (hereinafter referred to as “evaluation value”) is calculated as shown by Expression (13) described below.

[Math. 13]

P′(Sᵢ)=P(Sᵢ)·P(Tᵢ)·P(Tᵢ₊₁)·P(Tᵢ₊₂) (Expression 13)

“P(Sᵢ)” in Expression (13) described above is likelihood (hereinafter referred to as “parameter”) calculated based on the method explained in the first embodiment, that is, the difference between adjacent heartbeat intervals. “P(Tᵢ)” (hereinafter referred to “first probability value”), “P(Tᵢ₊₁)” (hereinafter referred to as “second probability value”), and “P(Tᵢ₊₂)” (hereinafter referred to as “third probability value”) in Expression (13) described above are calculation results of respective probability values for three peaks output by the method explained in the second embodiment, that is, the learned model.

In the calculation shown by Expression (13) described above, the parameter “P(Sᵢ)” is calculated based on the difference between a first heartbeat interval configured by a peak of the first probability value “P(Tᵢ)” and a peak of the second probability value “P(Tᵢ₊₁)” and a second heartbeat interval by a peak of the second probability value “P(Tᵢ₊₁)” and a peak of the third probability value “P(Tᵢ₊₂)”.

In the calculation shown by Expression (13) described above, likelihoods of the three peaks configuring the two heartbeat intervals are calculated by the method explained in the second embodiment and are set as the first probability value “P(Tᵢ)”, the second probability value “P(Tᵢ₊₁)”, and the third probability value “P(Tᵢ₊₂)”.

Therefore, the evaluation value “P′(Sᵢ)” is calculated by multiplying together the parameter “P(Sᵢ)”, the first probability value “P(Tᵢ)”, the second probability value “P(Tᵢ₊₁)”, and the third probability value “P(Tᵢ₊₂)”

The terms in Expression (13) described above are indicated as explained below.

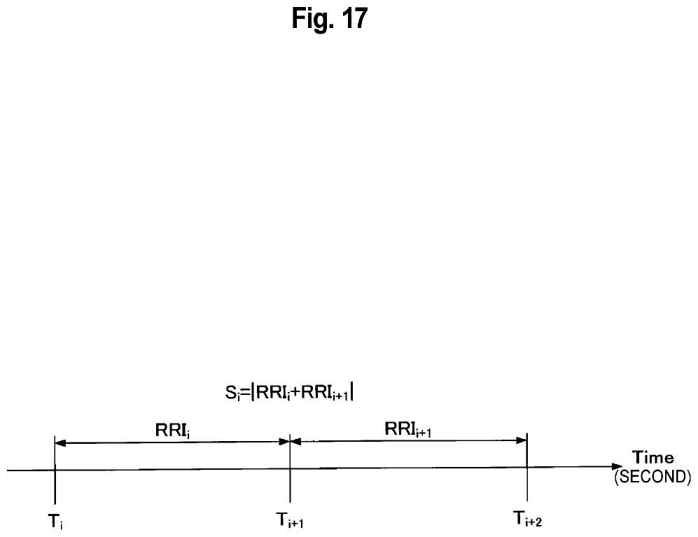

FIG. 17 is a diagram showing an example of heartbeat intervals and probability values in the third embodiment. For example, peaks occur in the order of “i”, “i+1”, “i+2”, In this example, a first heartbeat interval “RRIᵢ” includes, as a first peak and a second peak, peaks that occur at a point in time “Tᵢ” and a point in time “Tᵢ₊₁”. A second heartbeat interval “RRIᵢ₊₁” adjacent to the first heartbeat interval “RRIᵢ” is calculated using, as the first peak and the second peak, peaks that occur at the point in time “Tᵢ₊₁” and a point in time “Tᵢ₊₂”.

With such calculation, a selected sequence set “Xₛ”, which is the most likely sequence, is selected from the set of the differences of the adjacent RRIs shown in Expression (9) described above. Such selection can be shown as Expression (14) described below.

$$\begin{matrix}[ Math . 14 ] & \\ {X}_{S} = {argmax}_{{S}_{i}} {\prod }_{i = 1}^{M - 1} P ( {S}_{i} ) \cdot {\prod }_{i = 1}^{M + 1} P ( {T}_{i} ) = {argmin}_{{S}_{i}} \{ - {\sum }_{i = 1}^{M - 1} ln P ( {S}_{i} ) - {\sum }_{i = 1}^{M + 1} ln P ( {T}_{i} ) \} & ( Expression 14 )\end{matrix}$$

Note that “M” in Expression (14) described above represents the number of heartbeat intervals to be estimated. When the likelihoods of the peaks and the likelihoods by the differences between the adjacent heartbeat intervals are generally evaluated in this way, it is possible to accurately measure a heartbeat.

### Experiment Result

A result obtained by experimenting accuracy of the sequence selected by the method explained in the third embodiment is explained below.

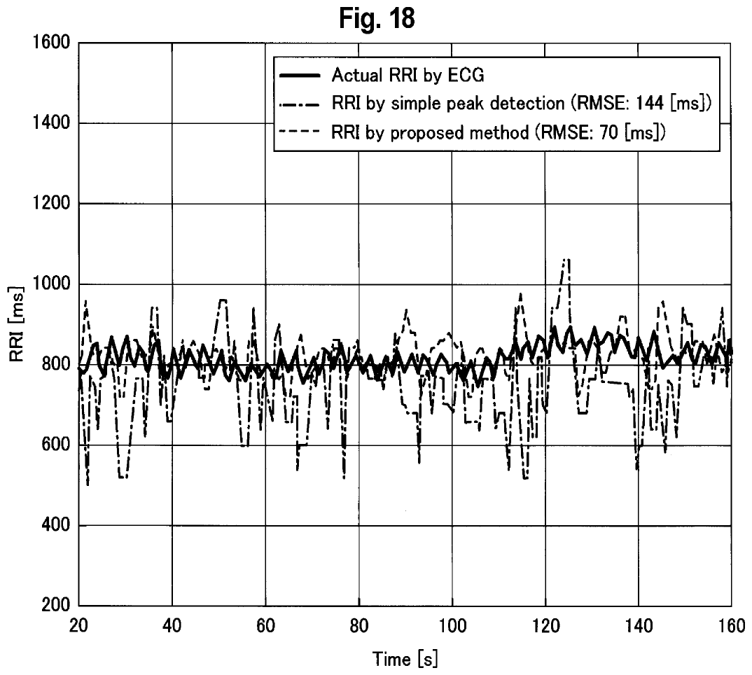

FIG. 18 is a diagram showing a first experiment result in the third embodiment.

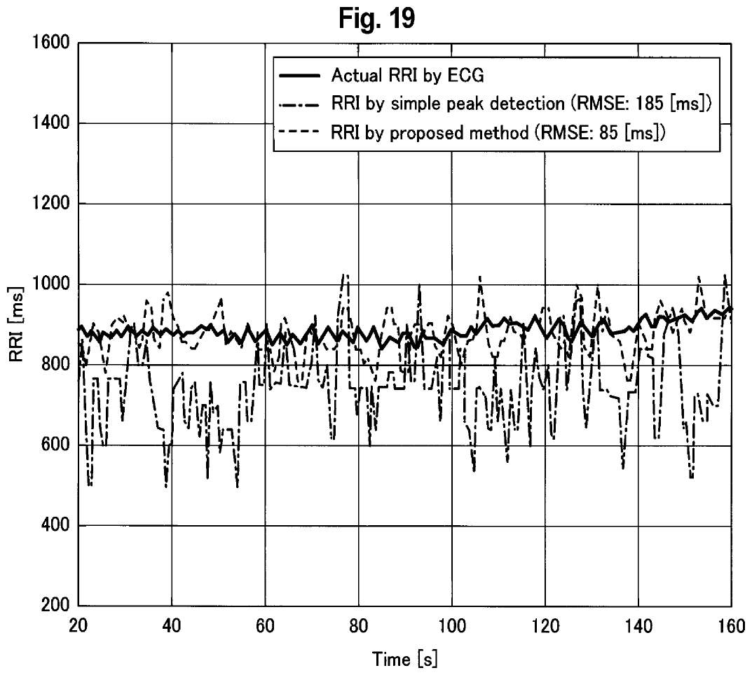

FIG. 19 is a diagram showing a second experiment result in the third embodiment.

In FIG. 18 and FIG. 19, subjects are different. FIG. 18 and FIG. 19 show measurement results of an electrocardiograph (hereinafter referred to as “ECG”) (“Actual RRI by ECG” in the figures indicate the measurement results by the electrocardiograph) as true values. In FIG. 18 and FIG. 19, results obtained by performing simple peak detection and calculating RRIs (indicated by “RRI by simple peak detection” in the figures) are shown as comparative examples. On the other hand, “RRI by proposed peak detection” in the figures is results obtained by calculating RRIs with the method explained in the third embodiment.

The comparison is performed by an RMSE (Root Mean Square Error) shown in Expression (15) described below.

$$\begin{matrix}[ Math . 15 ] & \\ RMSE = \sqrt{\frac{1}{N} {\sum }_{n = 1}^{N} {\vert {RRI}_{est} ( i ) - {RRI}_{ref} ( i ) \vert}^{2}} & ( Expression 15 )\end{matrix}$$

$$N : Number of observed RRIs$$

$${RRI}_{est} ( i ) : i - th estimated RRI$$

$${RRI}_{ref} ( i ) : True value of an i - th RRI$$

A value of the RMSE calculated by Expression (15) described above is an evaluation indicator indicating that, as the value is smaller, measurement closer to a value of the ECG can be performed. Whereas the value of the RMSE was “144 [ms]” and “185 [ms]” in the comparative examples, the value of the RMSE was “70 [ms]” and “85 [ms]” in the method explained in the third embodiment. It is possible to accurately measure a heartbeat when the heartbeat is measured by the method explained in the third embodiment in this way.

The evaluation value may be calculated by performing weighting as shown by Expression (16) described below.

[Math. 16]

P′(Sᵢ)=α·P(Sᵢ)·(1−α)·{P(Tᵢ)·P(Tᵢ₊₁)·P(Tᵢ₊₂) (Expression 16)

“α” in Expression (16) described above is a “weight coefficient”. Note that the other variables are the same as those of Expression (13) described above. Expression (16) described above is different compared with Expression (13) described above in that the parameter “P(Sᵢ)” is multiplied by the weight coefficient “α” and a probability value such as a first probability value is multiplied by “1−α”, which is a value obtained by subtracting the weight coefficient “α” from “1”.

Note that the weight coefficient “α” is set, for example, beforehand. As shown by Expression (16) described above, it may be able to be set by the weight coefficient “α” on which of a parameter based on a heartbeat interval and a probability value of a peak more importance is placed. For example, which of the parameter based on the heartbeat interval and the probability value of the peak important sometimes changes according to a state or the like of a subject. Even if such a change occurs, it is possible to accurately measure a heartbeat when the weight coefficient can be set as shown by Expression (16) described above.

### Fourth Embodiment

In a fourth embodiment, symmetry of a spectrum in the segment data SG is considered. In the following explanation, differences from the first embodiment and the second embodiment are mainly explained and redundant explanation is omitted. For example, an example is explained in which the segment data SG explained below is generated from the spectrogram SPE by the method explained in the second embodiment.

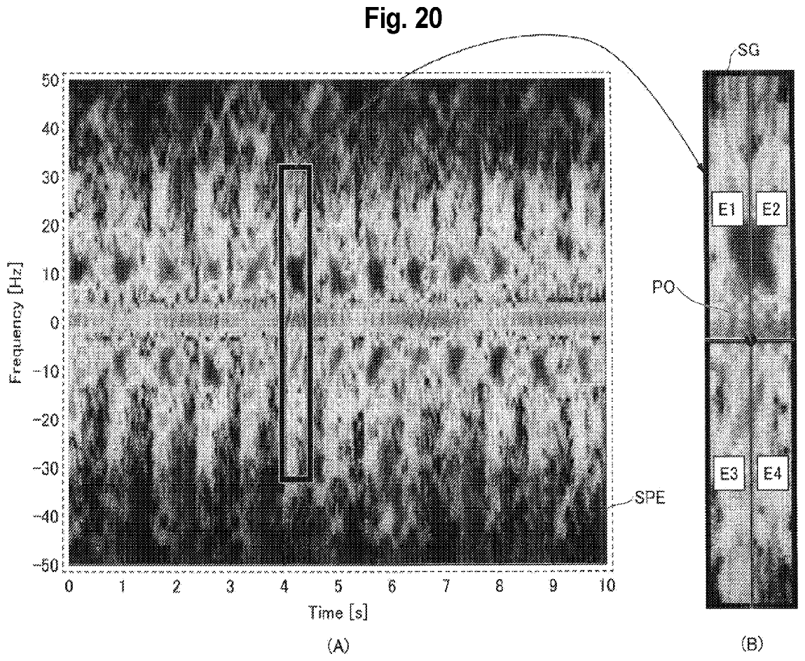

FIG. 20 is a diagram showing an example of a spectrogram and segment data in the fourth embodiment. Specifically, it is assumed that, in the spectrogram SPE shown in FIG. 20(A), the spectrogram SPE is divided and the segment data SG shown in FIG. 20(B) is generated. Note that a window is shifted along a time (in the figure, in the right direction) and the segment data SG is generated for each of windows.

Subsequently, as shown in FIG. 20(B), the segment data SG is equally divided by times (in the figure, lengths on the horizontal axis) and frequencies are divided by plus and minus (in the figure, divided at 0 Hz) to generate four regions. In the following explanation, the regions are referred to as “first region E1”, “second region E2”, “third region E3”, and “fourth region E4”.

When matrixes having powers of the first region to the fourth region as elements are represented as a “first matrix Q₁” to a “fourth matrix Q₄”, a time-series signal “s” can be calculated as shown by Expression (17) described below.

[Math. 17]

s=(Q₁.+Q₃).×fliplr(Q₂.+Q₄) (Expression 17)

In Expression (17) described above, “.+” represents addition of elements. “. x” represents integration of elements. Further, “fliplr” represents operation for reversing a column direction of a matrix. When the first region E1 to the fourth region are regarded as matrixes, numbers of rows of the first matrix Q₁ to the fourth matrix Q₄ coincide. Similarly, numbers of columns of the first matrix Q₁ to the fourth matrix Q₄ coincide. Therefore, when rows and columns are reversed by “fliplr”, reversals of a result obtained by adding up the first matrix Q₁ and the third matrix Q₃ and a result obtained by adding up the second matrix Q₂ and the fourth matrix Q₄ can be integrated.

If a region includes a heartbeat, the region has symmetry. Specifically, when the segment data SG includes a heartbeat, in FIG. 20(B), the segment data SG has a symmetrical distribution centering on an origin P0.

When the powers of the frequency components in the segment data SG have symmetry in this way, since two matrixes are added and integrated by the calculation of Expression (17) described above, a point where power occurs has a large value and is emphasized. Note that the emphasis is possible by only one of the addition and the integration. On the other hand, a portion not having symmetry has a small value. That is, a portion not due to a heartbeat can be attenuated. When the portion having symmetry is emphasized in this way, it is possible to accurately measure a heartbeat.

For example, when the calculation of Expression (17) described above is performed, a symmetrical portion included in the segment data SG can be emphasized. Specifically, the segment data SG is divided by absolute values of a time and a frequency to generate regions. When the regions generated in this way are set as matrixes and the matrixes are reversed, added up, and integrated, power of a frequency component having symmetry is emphasized.

### Experiment Result

An experiment result obtained by applying SIFT (Short Time Fourier Transform) with a window size set to “10 seconds” and a step size set to “one second” to the time series signal “s” calculated by Expression (17) described above to calculate a spectrogram is explained below. A result obtained by totaling, for each of frequencies, powers on respective spectrograms is also explained below.

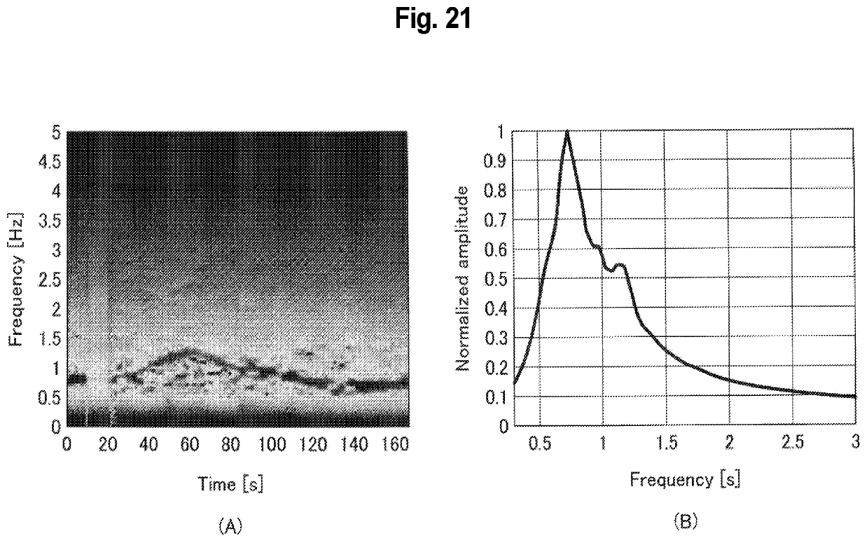

FIG. 21 is a diagram showing a first experiment result in the fourth embodiment.

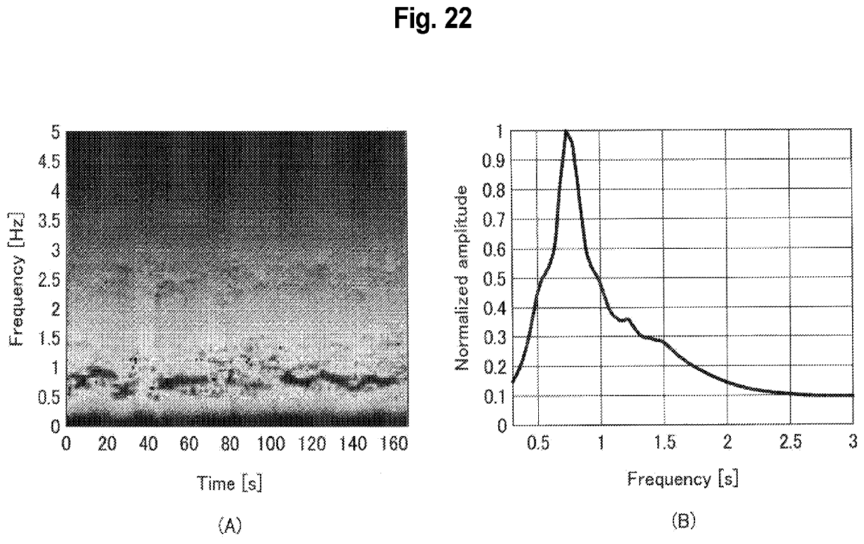

FIG. 22 is a diagram showing a second experiment result in the fourth embodiment.

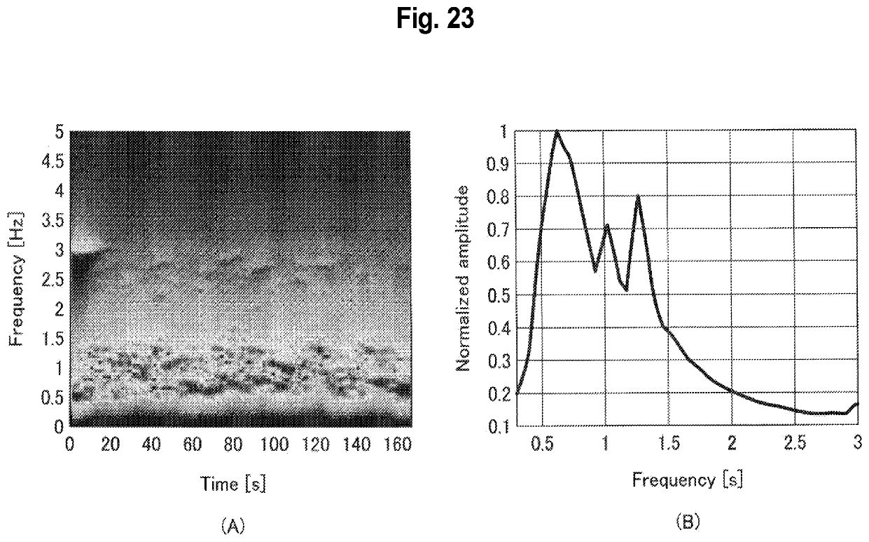

FIG. 23 is a diagram showing a third experiment result in the fourth embodiment.

In FIG. 21 to FIG. 23, subjects are different. (A) in the figures shows a calculation result of a spectrogram. (B) in the figures shows a result obtained by calculating an integrated value.

In experiments, measurement results by an ECG were measured as true values.

In a first experiment, the true value was 0.79 Hz.

In a second experiment, the true value was 0.75 Hz.

In a third experiment, the true value was 1.28 Hz.

All of results shown in respective (B)s are experiment results in which largest integrated values near the true values were large values and accurate measurement close to the true values was successfully performed.

An experiment for calculating an RRI was performed by a method using the fourth embodiment.

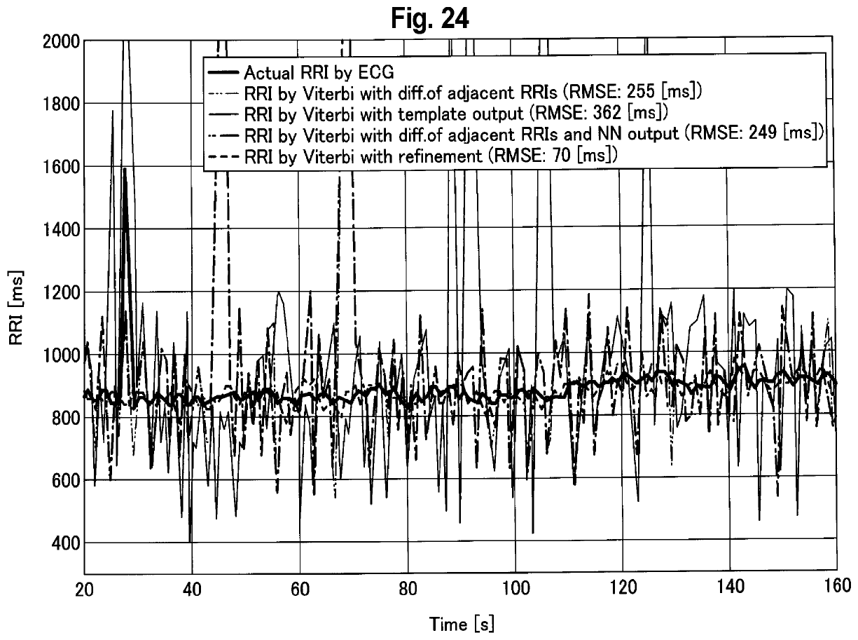

FIG. 24 is a diagram showing a first experiment result obtained by calculating a heartbeat interval in the fourth embodiment.

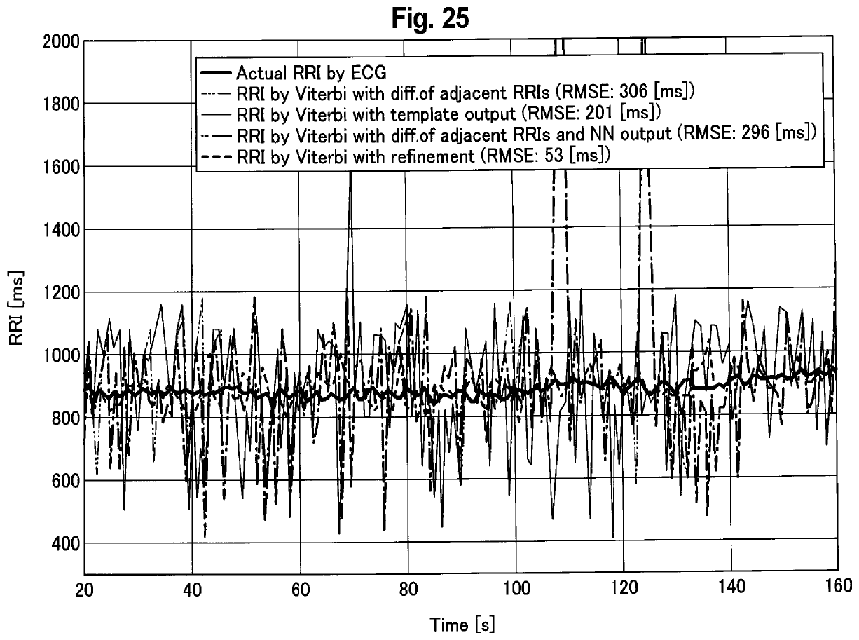

FIG. 25 is a diagram showing a second experiment result obtained by calculating a heartbeat interval in the fourth embodiment.

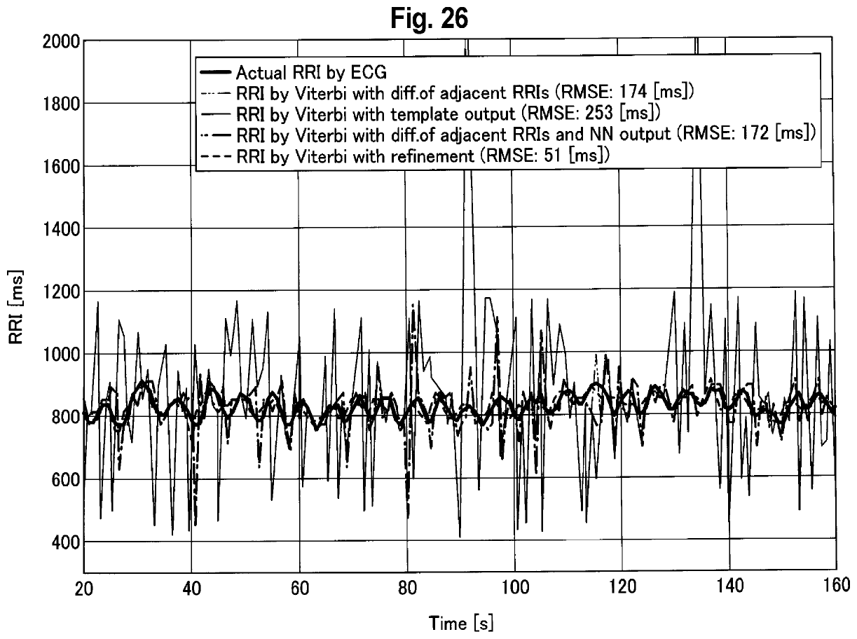

FIG. 26 is a diagram showing a third experiment result obtained by calculating a heartrate in the fourth embodiment.

In FIG. 24 to FIG. 26, a measurement result of an ECG is shown as a true value. The measurement result is “Actual RRI by ECG” in the figures.

As indicated by “RRI by Viterbi with refinement” in the figures, RMSEs were “70 ms”, “53 ms”, and “51 ms”. A heartbeat was successfully more accurately measured than other measurement results.

### Example of IQ Data Measured by a Doppler Radar

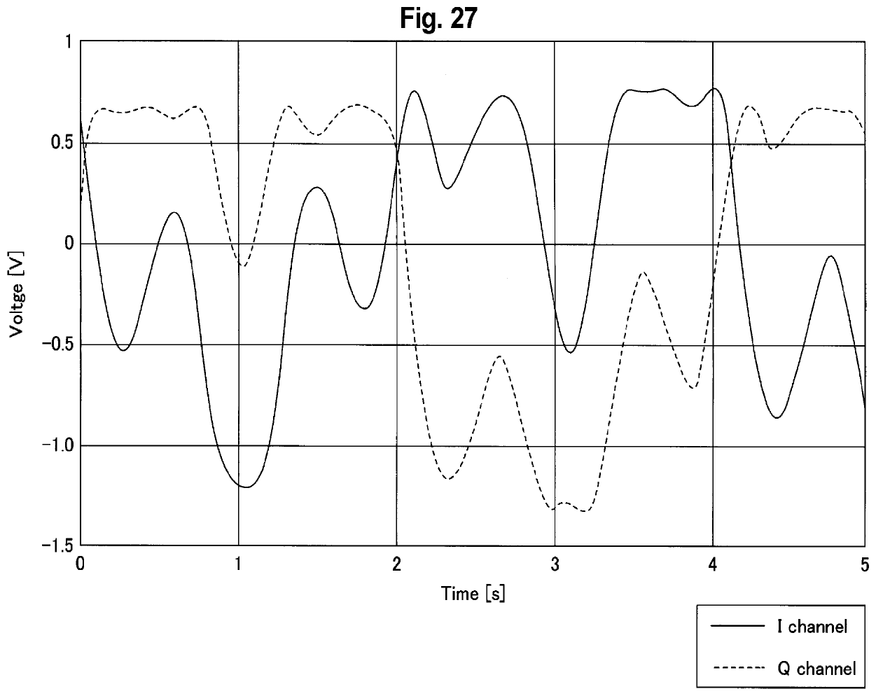

FIG. 27 is a diagram showing an example of IQ data measured by a Doppler radar. For example, the Doppler radar 12 outputs an illustrated signal. When arctan (Q/I) is calculated, a biological signal is obtained.

The Doppler radar 12 can measure, by irradiating a moving target object with a radio wave, a movement of the targe object can be measured based on a Doppler effect in which a frequency of a reflected wave changes. A configuration that can measure a movement of a subject in a noncontact manner in this way is desirable.

### Modifications

Note that the organism is not limited to a human and may be an animal or the like.

The learned model is used as a part of software in AI. Therefore, the learned model is a program. Therefore, the learned model may be distributed or executed via, for example, a recording medium or a network.

The learned model includes a network structure such as a CNN (Convolution Neural Network) or an RNN (Recurrent Neural Network). The learned model may be realized by the Cloud or the like that can be used via a network or the like.

As explained above, functional components may not include both of a component for “learning processing” and a component for “execution processing”. For example, in a stage where the “learning processing” is performed, the functional components may not include the component for “execution processing”. Similarly, in a stage where the “execution processing” is performed, the functional components may not include the component for “learning processing”. In this way, the functional components can be divided for the stages of “learning” and “execution” and formed as components excluding components different from the components for the processing to be performed. Note that, for example, after the “learning processing” or the “learning processing”, various settings in the network structure may be adjusted by the user.

### Other Embodiments

For example, the transmitter, the receiver, and the information processing devices may be pluralities of devices. That is, the processing and the control may be performed virtually, in parallel, decentrally, or redundantly. On the other hand, hardware may be integrated with or may function as the transmitter, the receiver, and the information processing device.

All or a part of the kinds of processing according to the present invention may be described in a low-level language such as assembler or a high-level language such as an object-oriented language and realized by a program for causing a computer to execute the biological detection method. That is, the program is a computer program for causing a computer such as an information processing device or a biological detection system to executes the kinds of processing.

Therefore, when the kinds of processing are executed based on the program, an arithmetic device and a control device included in the computer perform an arithmetic operation and control based on the program in order to execute the kinds of processing. A memory included in the computer stores data used for the processing based on the program in order to execute the kinds of processing.

The program can be recorded in a computer-readable recording medium and distributed. Note that the recording medium is a medium such as a magnetic tape, a flash memory, an optical disk, a magneto-optical disk, or a magnetic disk. Further, the program can be distributed through an electric communication line.

The preferred embodiments and the like are explained above. However, without being limited to the embodiments and the like explained above, various modifications and substitutions can be applied to the embodiments and the like explained above without departing from the scope described in the claims.

This international application claims the priority based on Japanese Patent Application No. 2020-140401 filed on Aug. 21, 2020, the entire content of Japanese Patent Application No. 2020-140401 being incorporated in this international application.

### REFERENCE SIGNS LIST

1 Biological detection system

2 Subject

10F1 Signal acquirer

10F2 Analyzer

10F3 Integrated value calculator

10F4 Peak specifier

10F5 Peak set generator

10F6 Selector

10F61 Heartbeat interval calculator

10F62 Difference calculator

10F7 Divider

10F8 Output unit

11 Amplifier

12 Doppler radar

12LNA Adjuster

12Rx Receiver

12S Source

12Tx Transmitter

13 Filter

BM12 Twelfth branch metric

BM13 Thirteenth branch metric

BM14 Fourteenth branch metric

BM23 Twenty-third branch metric

BM24 Twenty-fourth branch metric

BM25 Twenty-fifth branch metric

BM34 Thirty-fourth branch metric

DT Teacher data

E1 First region

E2 Second region

E3 Third region

E4 Fourth region

FR1 Heartbeat frequency component

FR2 Positive frequency component

FR3 Negative frequency component

HB Correct answer data

MDL Learned model

MDLB Learning model

P0 Origin

PKS1 First peak set

PKS2 Second peak set

PKS3 Third peak set

Q1 First matrix

Q2 Second matrix

Q3 Third matrix

Q4 Fourth matrix

SEC Sequence

SEC1 First section

SEC2 Second section

SG Segment data

SPE Spectrogram

T1 First peak generation time

T2 Second peak generation time

T12 Peak detection time

TR1 First point in time

TR2 Second point in time

TR3 Third point in time

1. A biological detection device comprising:
a signal acquirer that acquires a signal including a heartbeat;
an analyzer that performs a frequency analysis of the signal to generate a spectrogram;
an integrated value calculator that integrates energy of a heartbeat frequency component, which is a frequency component corresponding to the heartbeat, among frequency components of the signal shown by the spectrogram and calculates an integrated value;
a peak specifier that specifies peaks, which are maximum values, in the integrated value;
a peak set generator that combines, among the peaks, a first peak and a second peak, which occurs in a later time than the first peak, and generates a peak set; and
a selector that selects, from the peak set, a sequence having highest likelihood in a maximum likelihood estimation method.

2. The biological detection device according to claim 1, wherein the maximum likelihood estimation method is a Viterbi algorithm.

3. The biological detection device according to claim 2, wherein the selector calculates a branch metric representing likelihood between the first peak and the second peak and selects the sequence.

4. The biological detection device according to claim 3, further comprising:
a heartbeat interval calculator that calculates a first heartbeat interval, which is an interval of the heartbeat decided by the first peak and the second peak, and a second heartbeat interval, which is a next heartbeat of the first heartbeat interval; and
a difference calculator that calculates a difference between the first heartbeat interval and the second heartbeat interval, wherein
the selector selects the sequence in which a cumulative value obtained by totaling squared values of the difference is minimum.

5. A biological detection device comprising:
a signal acquirer that acquires a signal including a heartbeat;
an analyzer that performs a frequency analysis of the signal to generate a spectrogram;
a divider that generates segment data obtained by dividing the spectrogram in predetermined time units; and
an output unit that outputs, based on a learning model learned using the segment data as teacher data, a probability value of the segment data being data representing the heartbeat.

6. The biological detection device according to claim 4, further comprising:
a divider that generates segment data obtained by dividing the spectrogram in predetermined time units; and
an output unit that outputs, based on a learning model learned using the segment data as teacher data, a probability value of the segment data being data representing the heartbeat, wherein
the biological detection device calculates a first probability value, a second probability value, and a third probability value, which are the probability values of peaks configuring the first heartbeat interval and the second heartbeat interval,
calculates, based on the difference, a parameter representing likelihood, and
selects the sequence based on an evaluation value calculated by multiplying together the parameter, the first probability value, the second probability value, and the third probability value.

7. The biological detection device according to claim 1, wherein
the biological detection device generates segment data obtained by dividing the spectrogram in predetermined time units, and
emphasizes a symmetrical portion included in the segment data.

8. The biological detection device according to claim 1, wherein the signal acquirer acquires the signal with a Doppler radar.

9. A biological detection method performed by a biological detection device, the biological detection method comprising:
a signal acquisition procedure in which the biological detection device acquires a signal including a heartbeat;
an analysis procedure in which the biological detection device performs a frequency analysis of the signal to generate a spectrogram;
an integrated value calculation procedure in which the biological detection device integrates energy of a heartbeat frequency component, which is a frequency component corresponding to the heartbeat, among frequency components of the signal shown by the spectrogram and calculates an integrated value;
a peak specifying procedure in which the biological detection device specifies peaks, which are maximum values, in the integrated value;
a peak set generation procedure in which the biological detection device combines, among the peaks, a first peak and a second peak, which occurs in a later time than the first peak, and generates a peak set; and
a selection procedure in which the biological detection device selects, from the peak set, a sequence having highest likelihood in a maximum likelihood estimation method.

10. A program for causing a computer to execute the biological detection method according to claim 9.
